(19) United States
(12) Patent Application Publication
(10) Pub. No.: US 2025/0224394 A1
(43) Pub. Date: Jul. 10, 2025

(54) NOVEL TWO ELECTRODE-BASED CORRECTION APPROACH FOR ELIMINATION OF BIOFOULING FROM LABEL-FREE AFFINITY BIOSENSORS FOR DETECTION

(71) Applicant: UNIVERSITY OF PITTSBURGH - OF THE COMMONWEALTH SYSTEM OF HIGHER EDUCATION, 130 Thackeray Avenue, PA (US)

(72) Inventors: Prashant N. KUMTA, Pittsburgh, PA (US); Abhijit ROY, Mt. Lebanon, PA (US); Mitali S. PATIL, Silver Spring, MD (US); Moni Kanchan DATTA, Pittsburgh, PA (US); Sangeetha KUNJUKUNJU, Pittsburgh, PA (US)

(21) Appl. No.: 18/699,807

(22) PCT Filed: Oct. 11, 2022
(86) PCT No.: PCT/US2022/046216

§ 371 Date: Apr. 9, 2024

Related U.S. Application Data
(60) Provisional application No. 63/254,284, filed on Oct. 11, 2021.

(51) Int. Cl. G01N 33/543; G01N 33/68

(52) U.S. Cl. CPC G01N 33/5438; G01N 33/68

(57) ABSTRACT

The invention relates to a novel two-electrode approach that obviates non-specific adsorption (NSA) commonly termed, “biofouling” on biosensors. The unique two-electrode approach is based on baseline and sensing electrodes that are exposed to conditions with and without the detecting analyte. Proper corrections for normalization of the baseline and the intercept are made to eliminate the prevalence of biofouling and accurately detect the presence of the biomarkers without any interference. A corrective method nullifies the influence of any interfering factors present in animal and human blood, serum, biological body fluids or in any natural or synthetic solution that could bind to the label free biosensor (i.e., non-specific adsorption) and influence its sensitivity, accuracy, rapid detectability, specificity, precision, and reproducibility.

### CROSS-REFERENCE TO RELATED APPLICATIONS

This patent application claims priority under 35 U.S.C. 119 (e) from U.S. Provisional Patent Application No. 63/254,284, filed Oct. 11, 2021, entitled “NOVEL TWO ELECTRODE-BASED CORRECTION APPROACH FOR ELIMINATION OF BIOFOULING FROM LABEL-FREE AFFINITY BIOSENSORS FOR DETECTION OF BIOMARKERS FROM ANIMAL AND HUMAN BLOOD, SERUM, AND BODY FLUIDS”, the contents of which are incorporated herein by reference.

### FIELD OF THE INVENTION

The invention relates to a two electrode-based correction system and method for reducing or precluding the interference of biofouling from biosensor detection of biomarkers in bodily fluid samples including animal and human blood, serum, and bodily fluids. More particularly, the invention includes a baseline electrode without the sensing element for the biosensor or baseline biosensor and a sensing electrode containing all the functionalized components for the biosensor or a fully functionalized functional sensing biosensor.

### BACKGROUND

A biosensor is a bioanalytical device designed to detect or measure a chemical or biological substance by measuring the interaction between the target substance and a corresponding detection element and translating that interaction into a readable signal. This implies that techniques such as enzyme linked immunosorbent assays (ELISAs) and lateral flow devices (LFDs) can also be categorized as biosensors. However, recently developed biosensors focus on providing rapid results with minimal preparation and turnover time, such as glucose biosensors for measuring blood glucose (which operates in a matter of seconds), or over-the-counter pregnancy tests (which operates in a matter of minutes). A typical biosensor consists of the following components.
Analyte is the target molecule that the biosensor is intended to detect. Biosensors typically focus upon markers that are released into bodily fluids such as sweat, blood, urine, saliva, or other such biological fluids. Analytes can range from being molecules, electrolytes, proteins, antigens, ligands, and DNA strands to possibly complexes or even cells.
Biological detection element is the detection element that corresponds to the target analyte with strong selectivity and specificity to avoid interaction with other analytes in solution. The most common biological detection elements utilized in biosensors are enzymes, antibodies, and receptors, although aptamers have been widely utilized recently.
Bio/material interface is used to tether the biological detection element to a material interface that interacts favorably with the transducer, to translate the interaction between the analyte and the biological detection element into a readable signal. The interface is a material, such as metal (e.g., gold, silver, platinum, and titanium), chitosan graphene, carbon, silica, and polymers, that the biological detection element can directly bind to, or a material interface with biological tethering groups. The material interfaces may be nanoparticles, nanowires, or patterned metal on a non-conductive surface. The biological interfaces may be crosslinkers that tether the biological detection element directly to the surface, or even self-assembled monolayers (SAMs), which are molecular assemblies of molecules or proteins or DNA forming ordered domains spontaneously on surfaces via adsorption or chemical linkages.
Transducer is coupled with the bio/material interface to allow the interaction between the biological detection element and analyte to be “transduced” (translated) into a readable signal. Widely used transducers in biosensors are electrochemical (amperometry, potentiometry, conductimetry, impedimetry), optical (colorimetric, fluorescence, luminescence), and mass change (piezoelectric/acoustic wave). Electrochemical biosensors are most often used in point-of-care (POC) devices as they are portable, simple, reproducible, accurate, easy to use, rapid detection, cost-effective, and disposable. For instance, glucose biosensors are electrochemical biosensors based on screen-printed amperometric disposable electrodes. While amperometric and potentiometric transducers have been the most commonly used detection elements for biosensing devices, impedimetric devices have been increasingly utilized due to their classification as label-free detection biosensors.
Measuring device reads the transduced signal and translates the signal into an output that is transformed into the concentration or level of the analyte present in the sample. The measuring device is highly dependent on the transducer utilized. Many transducers are expensive, and in the case of optical sensors, are more suitable for laboratory-based testing rather than rapid, point-of-care devices.

Antibodies and enzymes are commonly utilized detection elements in biosensors due to their high affinity and specificity. However, there are disadvantages associated therewith—namely, antibodies and enzymes have relatively short shelf lives, are restricted by in vivo parameters, have batch-to-batch variation, and are sensitive to chemical or temperature changes. The use of synthetic aptamers (as compared to biological antibodies and enzymes) provides significant improvements for a biosensor. Aptamers are synthetic oligonucleotide sequences that are synthesized to bind to their target with high affinity and specificity, and therefore, provide significant improvements for a biosensor. The synthetic process of producing aptamers ensures the following properties and characteristics: high stability in various environments, long shelf lives, and minimal batch-to-batch variation, while maintaining their affinity and specificity. The aptamer-based biosensor can provide a stable system that may be more easily translated to a medical device. Furthermore, the small size and lack of hydrophobic core in aptamers can prevent aggregation, which has been found to be problematic in antibodies.

Synthesis of an impedimetric device requires a conductive material interface. There are several advantages associated with the use and selection of platinum as the material interface, such as its well-known chemically and electrochemically inert noble metal status, high electrical conductivity, and biocompatibility. However, there are also disadvantages associated with platinum, primarily its inert nature-most biosensor studies conducted on platinum interfaces still utilize non-chemically linked antibodies and enzymes as detection elements, and often use platinum electrodes or nanoparticles in tandem with other material interfaces, such as, carbon nanotubes/nanocomposites, graphene, chitosan, silica, polymers, or gold. It is not yet known in the art to develop an aptamer-based biosensor on a platinum interface alone.

Known biosensing devices are primarily used in a benchtop laboratory or in a clinical setting rather than a domestic setting. Various existing biosensor-based technologies are primarily based on fluorescent-immunoassays that require fluorescently labeled antibodies and a bench-top analyzer for the fluorescent assay. Common immunoassays include membrane-based immunoassays such as lateral flow devices (LFD) and enzyme-linked immunosorbent assays (ELISAs). These tests are highly dependent on the use of fluorescently labeled antibodies and spectrophotometers for analysis and detection of the fluorescence levels. The assays are typically conducted in laboratories and require significant preparation, pre-analytical time, and analytical time, which increases the overall turn-around time. In addition, many of these devices require a greater volume of blood than a typical glucose detector. In addition, since many of these devices require more blood than the typical glucose detector, hence they also require skilled personnel for patient blood drawing.

In general, there is a lack of standard diagnostic methods, turnover of processing blood samples in hospitals and laboratories is frequently slow, and common diagnostic methods are expensive, time-consuming, invasive, requiring the patient to be tested in a medical facility and requiring the results to be obtained by skilled and trained personnel, which do not promote routine testing. A lack of standard diagnostic methods and slow turnover for processing blood samples in hospital laboratories indicate the critical necessity of a point-of-care diagnostic biosensor for the rapid, accurate, reproducible, and sensitive detection of cardiac markers in blood.

Electrochemical impedance spectroscopy (EIS) is a technique that measures the impedance (opposition to the flow of alternating current, AC) of a system over a range of frequencies within an electrochemical cell, thus measuring the frequency response of the system. Impedance (Z) essentially translates the concept of resistance to an AC system and possesses both magnitude and phase due to the sinusoidal current, whereas resistance possesses only magnitude (resistance can be depicted as an impedance with zero phase angle). Impedimetric biosensors are highly sensitive and are label-free, thus significantly reduce the complexity of the system by removing the need for labels such as fluorescent tags. In addition, impedimetric biosensors are amenable to miniaturization and are cost-efficient, thus offering a low-cost path forward for rapid analysis. Therefore, impedimetric biosensors are ideal for point-of-care diagnostics and can be highly effective for direct use at the patient-bedside, in-ambulance use by paramedics, or even during clinical visits as a useful screening device.

According to the American Heart Association and National Health and Nutrition Examination Survey, approximately 121.5 million people in the U.S. suffered from some form of cardiovascular diseases (CVDs) in 2016, and the cost burden (both direct and indirect) of cardiovascular diseases exceeded $351.2 billion. By 2035, 45.1% of the US population is projected to have some form of CVD and between 2015 and 2035, the total direct medical costs of CVD are projected to escalate from $318 billion to $749 billion with the total indirect costs (attributable to lost productivity) for all fatal and nonfatal CVDs estimated to increase from $237 billion in 2015 to $368 billion in 2035. Further, CVDs and stroke accounted for 14% of the total U.S. health expenditures in 2014-2015, more than any major diagnostic group. Unfortunately, the prevalence and costs of cardiovascular diseases are projected to continue to spiral over the years despite CVDs being largely preventable due to the rise in incidences of obesity, hypertension, and diabetes. This high prevalence is due to CVDs being clinically silent with only non-specific symptom evidence until signs of serious complications arise, which has led to a lack of standard methods for CVD diagnosis. Delays in accurate diagnosis and treatment of CVDs are often associated with poor clinical outcomes and increased healthcare costs. Hence, it is imperative that a point-of-care (POC) device be developed for rapidly screening and monitoring of CVD and cardiomyopathy (CM) related heart failure (HF) risks to decrease incidence, deaths, and healthcare costs. Although many CMs are inherited, biochemical markers are a fundamental part of the diagnostic work-up and are useful in the prognostic assessment of the disease. The current diagnostic techniques for CVDs rely entirely on the use of expensive non-invasive imaging techniques, use of invasive methods, or on the timely and accurate interpretation of the physical symptoms experienced by patients. Unfortunately, current protocols dictate medical professionals treating any individual reporting chest pains accompanied by shortness of breath (dyspnea) (one of the most common symptoms of heart attacks) as potential acute myocardial infarction (AMI) patients. Therefore, resources are often constrained leading to situations where people with a milder form of CVDs or other unrelated diseases are also unnecessarily admitted and tested for possible heart attacks. However, in medical facilities with fewer resources, lack of these more sophisticated testing procedures could lead to possible misdiagnosis, thus potentially running the risk of treating patients for an entirely different condition rather than the real disease.

Label-free affinity biosensors (such as impedimetric biosensors) enable direct and real-time measurements of the chemical, physical and biological interactions among biomolecules and allows for rapid, reproducible, and accurate detection of chemical or biological species. Nanoscale sensors are predominantly surface-based and label-free to exploit inherent advantages of physical phenomena allowing high sensitivity without distortive labeling. There are three main criteria to be optimized in the design of surface-based and label-free biosensors: (i) the biomolecules of interest must bind with high affinity and selectively to the sensitive area; (ii) the biomolecules must be efficiently transported from the bulk solution to the sensor; and (iii) the transducer concept must be sufficiently sensitive to detect low coverage of captured biomolecules within reasonable time scales. Such biosensors show immense promise for bioanalytical applications, especially in medical diagnostics, where use of point-of-care (POC) biosensors are extremely valuable in various hospital, emergency, and residential patient care settings.

In contrast, with the current gold standard ELISA (enzyme-linked immunosorbent assay) assays, the label-free biosensors detect the target analytes directly, thus eliminating the need for multi-step assays or additional reagents. However, a disadvantage to label-free biosensors is that because they are based on measurements caused by the direct binding of analyte molecules to the biological detection element immobilized on the biosensor surface, their performance may be almost always compromised by interfering effects. Perhaps one of the strongest interferences, especially in biological samples, is the non-specific adsorption (NSA) of molecules on the biosensor surface, especially in whole blood and plasma, which is commonly and universally termed as “biofouling”. NSA is a persistent problem that negatively affects biosensors, decreasing sensitivity, specificity, and reproducibility. Passive and active removal methods exist to remedy this issue, by coating the surface or generating surface forces to shear away weakly adhered biomolecules, respectively. While many coatings have been developed to prevent biofouling, these coatings can deteriorate quickly, especially after the attachment of the biological detection elements. Therefore, the continual perennial search to reduce biofouling continues and is one of the most critical barriers to universal adoption and implementation of all types of biosensors in hospital, residential and emergency settings.

This invention is directed to a different approach than traditional biofouling related approaches that attempt to mitigate or prevent biofouling. Instead of reducing biofouling, this invention relegates the biofouling to the background noise/interference and completely normalizes the interference sufficiently and efficiently to be able to detect the target antigen in a wide array of samples. Additionally, this approach includes improved impedimetric aptasensors capable of providing one or more of efficient, early, reliable, convenient (e.g., point-of-care, on-demand), inexpensive, rapid, minimally or non-invasive, reproducible, sensitive, and accurate impedimetric detection and screening of biomarkers to provide preventative medication, monitoring, and therapeutic treatment for achieving favorable outcomes, which may be utilized outside the confines of a hospital or other medical facility, e.g., in a domestic setting, such as, a patient's home.

### SUMMARY OF THE INVENTION

An aspect of the invention provides a biosensor system to detect biomarkers of interest in a patient and reduce or preclude non-biomarker interference, including a baseline biosensor absent of a sensing element that includes an electrically conductive material interface having a surface, an immobilization agent applied or deposited onto the surface of the conductive material interface; and biotin conjugated with the immobilization agent; a fully functionalized functional sensing biosensor, which includes a conductive material interface having a surface, and a biological sensor agent applied to the surface of the conductive material interface, the biological sensor agent including an immobilization agent; and at least one aptamer or antibody selected to interact with the immobilization agent and selected to bind with specific biomarkers of interest; a signaling agent comprising an electrochemical impedance signal generated by binding of the aptamer or antibody with the specific biomarkers of interest; and a bodily fluid sample derived from the patient and in contact with the biotin of the baseline biosensor and at least one aptamer or antibody of the fully functionalized functional sensing biosensor, wherein the baseline biosensor detects interference in the bodily fluid sample, and the fully functionalized functional sensing biosensor detects a presence of the specific biomarkers of interest in the bodily fluid sample; and a correction applied to the baseline biosensor and the fully functionalized functional sensing biosensor to reduce or preclude any non-biomarker interference from the bodily fluid sample.

In certain embodiments, the bodily fluid sample is selected from blood, serum and urine samples of animal or human. In certain embodiments, the biomarkers are selected from C-reactive protein, Creatinine Kinase, Creatinine Phosphokinase (CPK), TroponinT, Myoglobin, IL-6, IL-18, Brain Natriuretic Peptide, and D-Dimer, immunosuppression drugs (ISDs), Tacrolimus; Sirolimus, Tau protein, glial fibrillary acidic protein, and breakdown products (GFAP-BDP) and ubiquitin C-terminal hydrolase L1 (UCH-L1).

The immobilization agent can be a binding agent. The binding agent can be selected from the group consisting of avidin, streptavidin, neutravidin and mixtures thereof.

The conductive material interface can include a multi-array of vertically aligned platinum wires. The multi-array of vertically aligned platinum wires can be arranged in a circular configuration.

In certain embodiments, the aptamer is effective to impedimetrically detect simultaneously a plurality of biomarkers in the bodily fluid sample.

Another aspect of the invention provides a biosensing method of detecting biomarkers of interest in a bodily fluid sample of a patient and to reduce or preclude any non-biomarker interference, including obtaining the bodily fluid sample from the patient; forming a detection device that includes a baseline biosensor absent of a sensing element, including a conductive material interface having a surface, an immobilization agent applied or deposited onto the surface of the conductive material interface; and biotin conjugated with the immobilization agent; a fully functionalized functional sensing biosensor, which includes a conductive material interface having a surface, and a biological sensor agent applied to the surface of the conductive material interface, the biological sensor agent including an immobilization agent; and at least one aptamer or antibody selected to interact with the immobilization agent and selected to bind with the biomarkers of interest; contacting the biotin of the baseline biosensor and the aptamer or antibody of the fully functionalized functional sensing biosensor with the bodily fluid sample; generating an electrochemical impedance signal for each of the baseline biosensor and the fully functionalized functional sensing biosensor; and applying a correction to the fully functionalized functional sensing biosensor based on the baseline biosensor to reduce or preclude any non-biomarker interference from the bodily sample.

In certain embodiments, the electrochemical impedance signal is transduced to a read-out value. In certain embodiments, the electrochemical impedance signal is also connected to a portable device such as a hand-held device that is effective to display the read-out value.

In certain embodiments, the detection device is in the form of a test strip and the method includes contacting the bodily fluid sample with the test strip; assessing a visual change to the test strip; correlating the visual change with a chart or key; and based on the said correlating, determining if the visual change is indicative of the presence of a change in electrochemical impedance and the presence of the cardiac biomarkers in the bodily fluid sample. In certain embodiments, the visual change is a color change.

The electrochemical impedance signal can be generated as a result of the biomarker of interest interacting with the aptamer.

Prior to applying the immobilization agent, the surface of the conductive interface material can be treated with a thiol-based compound. The thiol-based compound can be an aminothiol selected from cysteamine and/or glutaraldehyde.

### BRIEF DESCRIPTION OF THE DRA WINGS

A further understanding of the invention can be gained from the following description of the preferred embodiments when read in conjunction with the accompanying drawings.

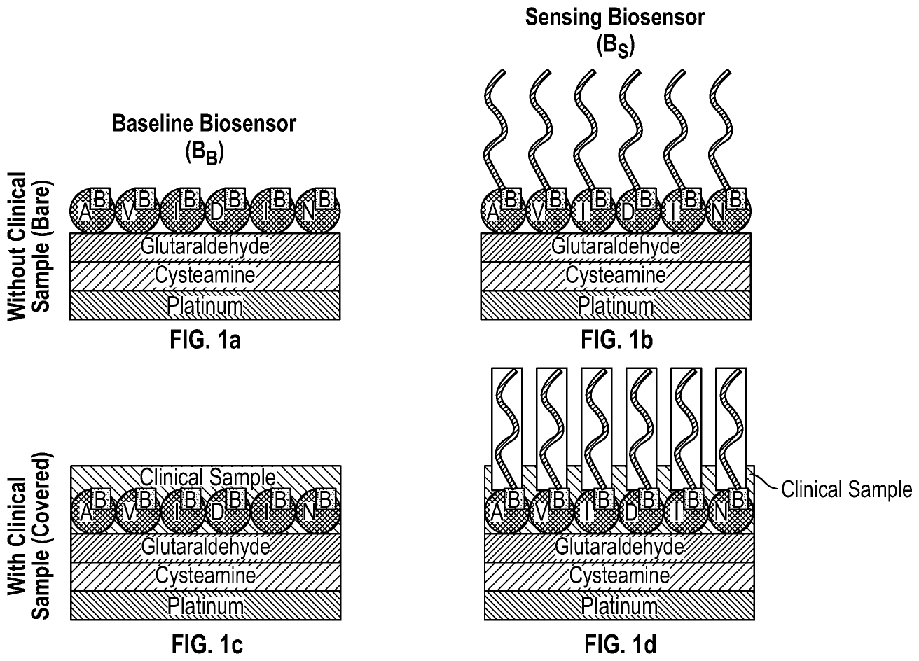

FIG. 1a is a schematic that depicts the baseline biosensor, in accordance with certain embodiments of the invention;

FIG. 1b is a schematic that depicts the sensing biosensor, in accordance with certain embodiments of the invention;

FIG. 1c is a schematic that depicts the baseline biosensor of FIG. 1a with a clinical sample, in accordance with certain embodiments of the invention;

FIG. 1d is a schematic that depicts the sensing biosensor of FIG. 1b with a clinical sample, in accordance with certain embodiments of the invention;

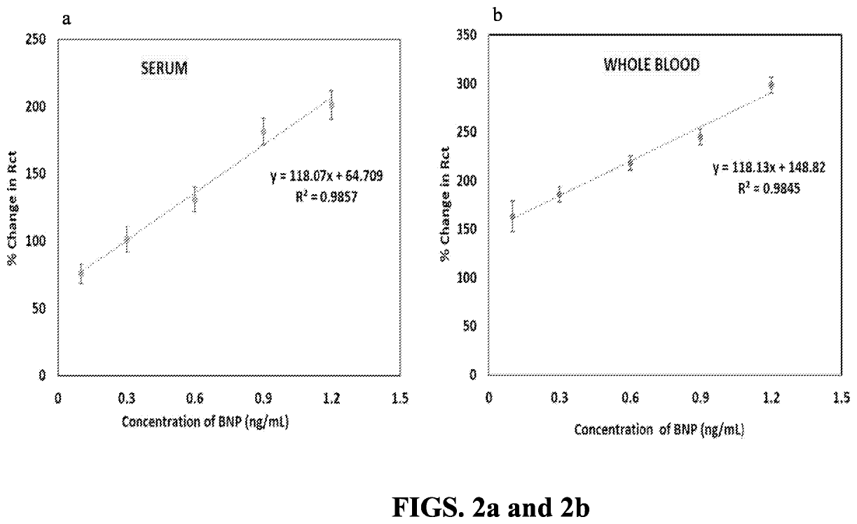

FIGS. 2a and 2b are calibration curves for BNP biosensors for serum and whole blood, respectively, with 5 m-Ret, in accordance with certain embodiments of the invention;

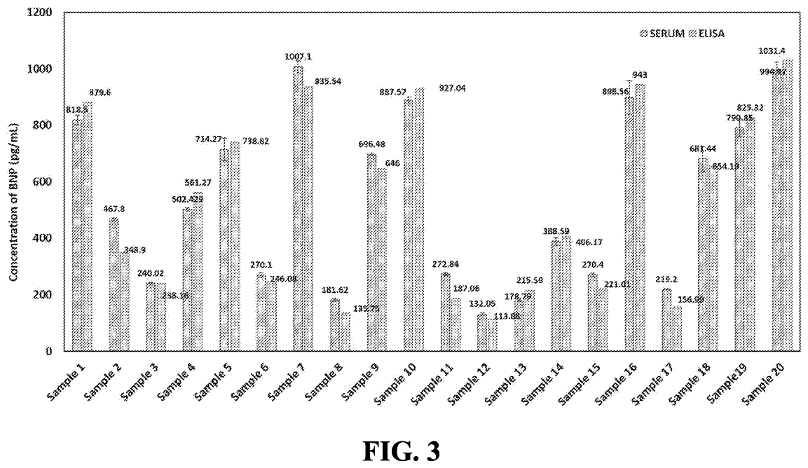

FIG. 3 is a graph that shows results of a human serum sample analysis using the two-electrode approach, wherein twenty serum samples were tested for BNP antigen and the change in Ret values were measured after five minutes of incubation time with the serum samples, in accordance with certain embodiments of the invention;

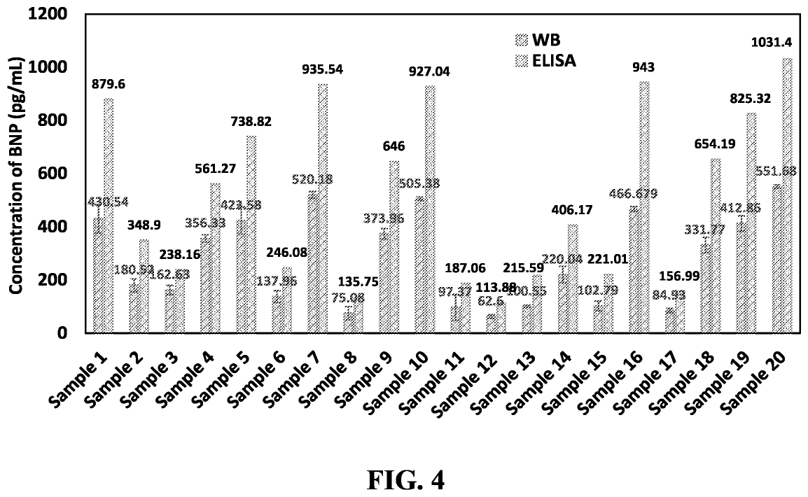

FIG. 4 is a graph that shows results of a human whole blood (WB) sample analysis using the two-electrode approach, wherein twenty whole blood samples were tested for BNP antigen and the change in R_{ct} values were measured after five minutes of incubation time with the whole blood samples, in accordance with certain embodiments of the invention;

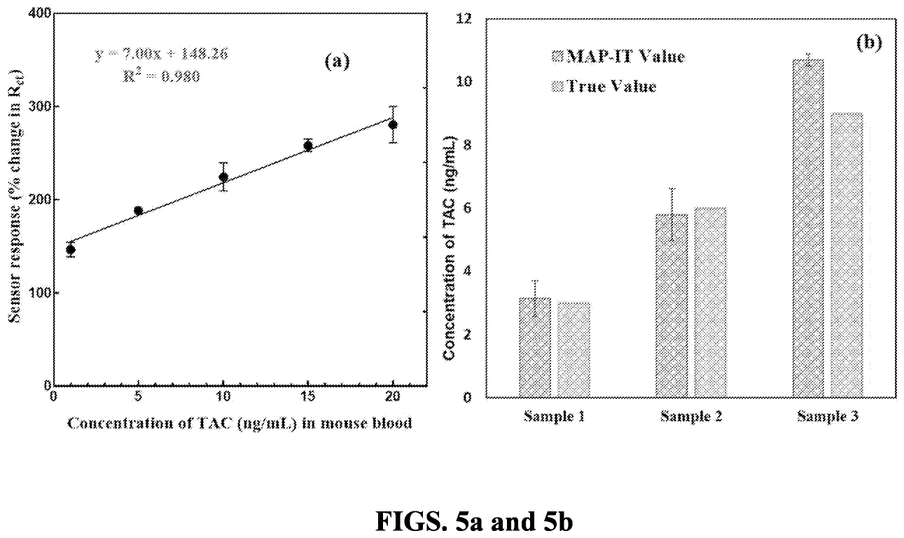

FIG. 5a is a calibration plot of biotin linked TAC binding protein related protein antibody biosensor dissolved in mouse blood, in accordance with certain embodiments of the invention;

FIG. 5b is a graph showing detection accuracy of TAC concentrations in blinded mouse blood samples (n=3), in accordance with certain embodiments of the invention;

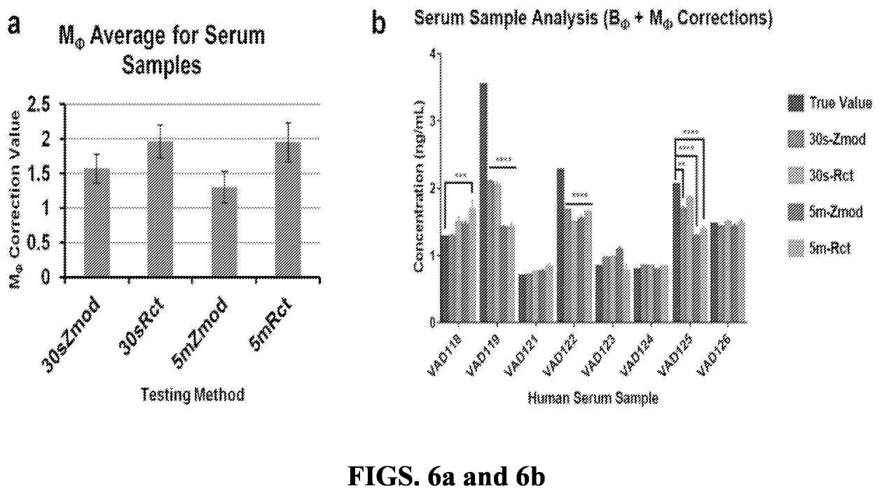

FIG. 6a is a graph showing MΦ average results for human serum samples (error bars represent ±SEM for n=8), in accordance with certain embodiments of the invention;

FIG. 6b is a graph showing recalculated measurements using both MΦ and BΦ corrections wherein asterisks stand for p-values calculated by 2-way ANOVA with post-hoc Dunnett's multiple comparisons against a control (true value), in accordance with certain embodiments of the invention;

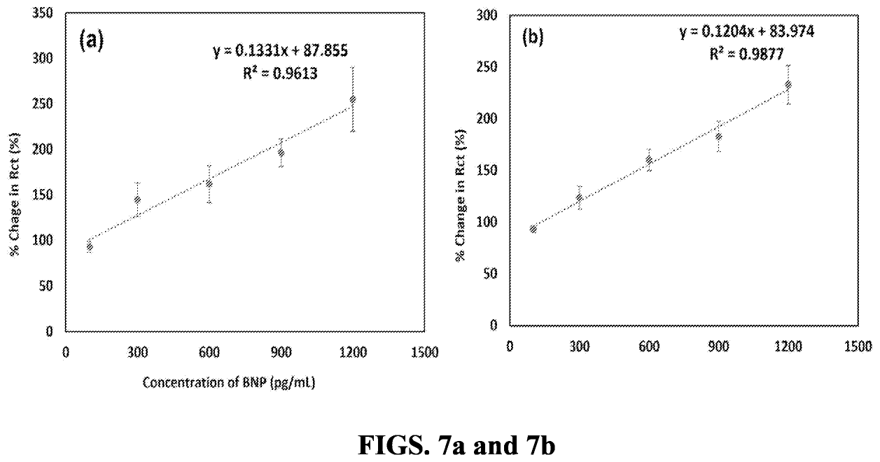

FIG. 7a is a plot showing the calibration curve prepared for detecting BNP in human serum by Method 1, in accordance with certain embodiments of the invention;

FIG. 7b is a plot showing the calibration curve prepared for detecting BNP in human whole blood samples by Method 1, in accordance with certain embodiments of the invention;

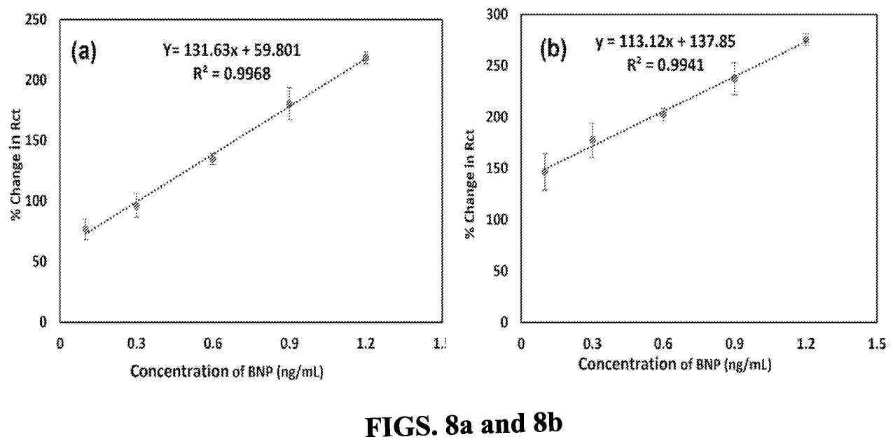

FIG. 8a is a plot showing the calibration curve prepared for detecting BNP in human serum by Method 2, in accordance with certain embodiments of the invention;

FIG. 8b is a plot showing the calibration curve prepared for detecting BNP in human whole blood samples by Method 2, in accordance with certain embodiments of the invention;

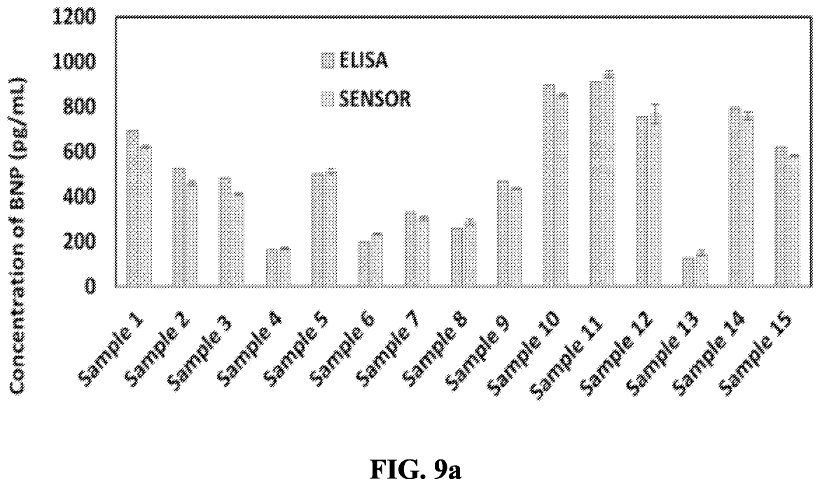

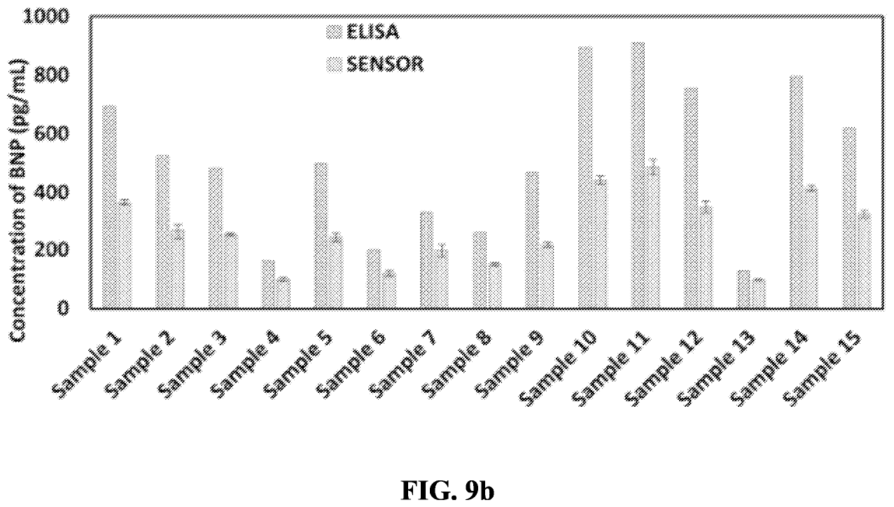

FIG. 9a is a graph showing the concentration of brain natriuretic peptide (BNP) in blinded unknown human serum samples obtained utilizing the inventive two-electrode detection assembly, in accordance with certain embodiments of the invention; and

FIG. 9b is a graph showing the concentration of BNP in blinded unknown human whole blood samples obtained utilizing the inventive two-electrode detection assembly, in accordance with certain embodiments of the invention.

### DETAILED DESCRIPTION OF THE PREFERRED EMBODIMENTS

The invention relates to a novel two-electrode approach that obviates non-specific adsorption (NSA) commonly termed, “biofouling”. The two-electrode approach is based on the baseline and fully functionalized functional sensing electrodes that are exposed to conditions with and without the detecting analyte. Proper corrections are then made to eliminate the prevalence of any biofouling. The invention provides a corrective method which can nullify the influence of any interfering factors present in the animal and human blood, serum, biological body fluids or in any natural or synthetic solution that could bind to the label free biosensor (i.e., non-specific adsorption) and influence its sensitivity, rapid detectability, accuracy, specificity, precision, and reproducibility. The invention accounts for the influence of various biological factors present in the blood, serum, or biological fluid on the sensor response. The method is universal, versatile, and applicable to any electrochemical biosensors including but not limited to impedimetric, potentiometric, amperometric, field effect transistor, voltametric biosensors as well as any other biosensing platform utilizing various detection and sensing mechanisms based on optical, electrical, electrochemical, ultrasound, magnetic or any other physical, chemical, physico-chemical and biological, as well as physico-chemico-biological changes. The method is independent of any physicochemical nature of the analyte and is useful and effective in the detection of any inorganics, organics, and biological molecules including drugs, proteins, serum, DNA, RNA, electrolytes, antibiotics, and vitamins. The method is reliable for the detection of cardiovascular disease (CVD), traumatic brain injury (TBI), and mitochondrial diseases (MDs) related biomarkers as well as detection of immunosuppression drugs (ISDs) in the case of vascularized composite allotransplant (VCA) patients who have received organ transplants. The corrective method is applicable to eliminate any biofouling-related interference in a biosensor signal, e.g., for cardiovascular disease related biomarkers, brain natriuretic peptide (BNP) and Troponin T (TnT) in various biological fluids including animal and human whole blood and serum.

As used herein with respect to the sensing electrode or sensing biosensor, the term “fully functionalized functional” means fabrication of the electrode or biosensor with all the chemical components added as self-assembled monolayers (SAM) to the electrode or biosensor of platinum in sequential form. These include but are not limited to cysteamine, glutaraldehyde, Neutravidin and biotinylated aptamer or antibody.

The corrective method includes at least two electrodes, wherein one electrode serves as the fully functionalized functional sensing electrode or sensing biosensor (B_{S}) and the other electrode serves as the baseline electrode or baseline biosensor (B_{B}). The method is useful for any number of electrodes or biosensors as long as at least one electrode or biosensor acts as the primary fully functionalized sensing electrode or fully functionalized functional sensing biosensor and at least one of the electrodes or biosensors acts as the baseline electrode or baseline biosensor. The method is independent of any physicochemical nature of the electrodes or biosensors and solely relies on the charge in charge transfer/impedance values of the detection parameter measured from the fully functionalized functional sensing and baseline electrodes or biosensors.

FIG. 1 is a schematic that illustrates a two-electrode biosensing model in accordance with certain embodiments of the invention. FIG. 1a shows the baseline biosensor (B_{B}) and FIG. 1b shows the fully functionalized functional sensing biosensor (B_{S}) without clinical sample deposition (i.e., bare B_{B} and bare B_{S}); FIG. 1c shows the B_{B} and FIG. 1d shows the fully functionalized functional B_{S} with clinical sample deposition (i.e., covered B_{B} and covered B_{S}). The baseline biosensor is functionalized with biotin only (shown in FIG. 1a as “B” in a square box), i.e., the B_{B} is not functionalized with biotinylated aptamer of antibody following avidin (e.g., Neutravidin) binding; thus, ensuring that the baseline biosensor does not detect the target antigen and only detects interference from the biological sample. Whereas, the fully functionalized functional sensing biosensor is functionalized with biotinylated aptamer or antibody (shown in FIG. 1b as multiple curved lines) in addition to being functionalized with biotin (shown in FIG. 1b as “B” in a square box); thus, by this functionalization, enabling the fully functionalized functional B_{S} to detect not only the interference from the biological sample, but also the target antigen. It is essential for all the biotin sites on avidin (e.g., Neutravidin) to be bound by either biotin (in the case of the baseline biosensor) or biotinylated aptamer or antibody (in the case of the fully functionalized functional sensing biosensor). When each of the baseline and fully functionalized functional sensing biosensors is contacted with a clinical sample, the baseline biosensor detects the interference from the clinical sample, whereas the fully functionalized functional sensing biosensor detects the target antigen and the interference from the clinical sample. The fully functionalized functional sensing biosensor is then compared with the baseline biosensor, and the impact of the interference as detected by the baseline biosensor is completely taken into full account to determine the detection by the fully functionalized functional sensing biosensor of the target antigen, i.e., absent of the interference.

According to certain embodiments of the invention, the correction method entails essentially using a functionalized baseline biosensor electrode (as shown in FIG. 1a) that includes a bare platinum (P) electrode functionalized by, for example, self-assembled monolayers (SAM) of cysteamine (C) and glutaraldehyde (G), and Avidin (Neutravidin (N)), without the biotinylated aptamer or antibody serving as electrode 1 (the base electrode/biosensor); and the second electrode (electrode 2 or the fully functionalized functional sensor electrode/biosensor) including a bare platinum (P) electrode functionalized by self-assembled monolayers (SAM) of cysteamine (C) and glutaraldehyde (G), Avidin (e.g., Neutravidin (N)) and biotinylated aptamer (A) (as shown in FIG. 1b). Both electrodes 1 and 2 are then exposed to the same quantity of clinically relevant whole blood or serum sample. A comparison is made of the two electrodes' measurements of the impedances, R_{ct}, by the electrochemical impedance spectroscopy (EIS) method determined by the calibration curve of:

$$y = m * x + b \qquad ( Equation - 1 )$$

wherein y=% Δ in R_{ct} or Z_{mod} between the initial R_{ct} or Z_{mod} of the biosensor and the R_{ct} or Z_{mod} of the biosensor after exposure to the clinical sample. This accounts for correction or normalization of the baseline of the two electrodes, i.e., one without the biotinylated aptamer or antibody (B_{B}) and the second with the biotinylated aptamer or antibody (B_{S}), which serves to eliminate the non-specific adsorption (NSA) of any biological molecules, serum or peptides present in the clinically relevant sample. The clinically relevant unknown sample applied to each of the electrodes without the biotinylated aptamer (electrode 1) and with the biotinylated aptamer (electrode 2) has identical non-specific adsorbing or binding molecules and thus, they all essentially bind completely to the same extent on both the electrodes. However, in addition to binding to all the other binding sites presented by the platinum functionalized with cysteamine, glutaraldehyde, Neutravidin and biotinylated aptamer, the second electrode also only specifically binds to the relevant specific markers in the blood or serum specifically and selectively active only to the biotinylated aptamer or antibody. Therefore, by comparing the two electrodes, electrode 1 without the aptamer or antibody and electrode 2 with the aptamer or antibody, exposed to an identical quantity of whole blood or serum, the non-specific binding of proteins, signaling molecules, and other biological factors are completely accounted for giving a measure of the unknown amounts of the specific markers relevant to the aptamer or antibody in the whole blood or serum. That is, the non-specific binding of proteins and other biological factors will be the same for electrodes 1 and 2. Since electrode 2 will also having binding of the specific markers in the whole blood or serum sample, the measure of electrode 1 that is representative of the non-specific binding of proteins and other biological factors (for electrodes 1 and 2) is subtracted from the measure of electrode 2 that is representative of the non-specific binding of proteins and other biological factors as well as the binding of specific markers of the whole blood or serum sample, to unequivocally provide the exact measure of the specific markers only.

Normalization for the baseline of electrode 1 without the aptamer or antibody and electrode 2 with the aptamer or antibody without exposure to any clinically relevant blood or serum fluids combined with adequate correction of the intercept for the two electrodes, electrode 1 without the aptamer or antibody and electrode 2 with the aptamer or antibody when exposed to whole blood or serum, provides values of the true intercept which is applied to the calibration straight line curve to determine the exact concentration of the relevant marker present in the clinically relevant sample; thus serving to detect the unknown amounts of the specific markers in the clinically relevant whole blood or serum sample. That is, normalization is precisely conducted to account for any differences between electrode 1 and electrode 2 that include the bare platinum (P) functionalized with, e.g., self-assembled monolayers (SAM) of cysteamine (C) and glutaraldehyde (G), and Avidin (Neutravidin (N)). This is done by using the normalized baseline value for the sensing electrode 2 containing the biotinylated aptamer or antibody in presence of whole blood or serum, wherein the signal of electrode 2 in presence of whole blood or serum is modified by the ratio of the signal of electrode 1 without any biological fluids or blood and electrode 2 in absence of biological fluids or blood. That is, the resultant correction factor for the intercept and the normalized baseline value for the sensing electrode 2 is applied to electrode 1 when exposed to blood or serum containing unknown amounts of the specific marker to be measured and electrode 1 without exposure to blood or serum devoid of any markers to give the true intercept. Similarly, electrode 2 values are obtained when exposed to blood or serum containing unknown amounts of specific markers, and electrode 2 values obtained without exposure to blood or serum devoid of any markers. These two values are then utilized to obtain the unknown values of the specific marker using the value of the slope, m in Equation 1 obtained from the calibration curves.

The correlation between the two biosensors, i.e., electrodes 1 and 2, is represented by B_{Φ}:

$${B}_{Φ} = ( \% Δ {B}_{S} ) / ( \% Δ {B}_{B} ) \qquad ( Equation - 2 )$$

wherein % Δ B_{S}=% Δ R_{ct} or Z_{mod} between electrode 2 (biotinylated aptamer, B_{S}) with and without the biological blood/serum/fluids and the biological blood/serum/fluids is devoid of any specific markers; and % Δ B_{B}=% Δ R_{ct} or Z_{mod} between electrode 1 (biotin only, B_{B}) with and without the biological blood/serum/fluids and the biological blood/serum/fluids is devoid of any specific markers. Therefore, B_{Φ} is essentially the ratio between the y-axis intercept for the B_{S} biosensor to the B_{B} biosensor, where the y-intercept is presented as % ΔR_{ct} or Z_{mod} (the percent change between the signal of the biosensors with and without whole blood). Thus, in this embodiment, B_{Φ} represents the coverage of Neutravidin by the biological sample. Thus, B_{Φ} is represented as:

$${B}_{Φ} = {Area}_{exposed} ( {A}_{e} ) \div Total Area ( A ) = {A}_{e} \div A \qquad ( Equation - 3 )$$

Substituting Equation-2 with the layer thickness of the blood- and without blood-containing layers and solution resistivity results in Equation-3. Thus, B_{Φ} is also represented as:

$${B}_{Φ} = ( {B}_{B} - {B}_{N} ) / {B}_{S} \qquad ( Equation - 4 )$$

wherein B_{N} is the signal measured from the baseline of the biotin-only electrode 1 without any biological fluids or bloods, B_{B} is the signal of the biotin-only electrode 1 in the presence of blood/serum/biological fluids, and B_{S} is the signal of the biotinylated aptamer electrode 2 in the presence of blood/serum/biological fluids. The B_{S} signal was further normalized according to the following equation:

$${B}_{S}^{N} = ( {B}_{N} * {B}_{S} ) / {B}_{SB} \qquad ( Equation - 5 )$$

wherein B_{S}^{N} is the normalized signal of the biotinylated aptamer electrode 2 in the presence of blood/serum/biological fluids, and B_{SB} is the signal measured from the baseline of biotinylated aptamer electrode 2 without any biological fluids or bloods.

The true intercept of the B_{S} biosensor (electrode 2) is calculated using the B_{B} biosensor (electrode 1) and the average B_{Φ} values; Equation-2 can be modified as follows:

$$\% Δ {B}_{S} = {B}_{Φ} \times \% Δ {B}_{B} \qquad ( Equation - 6 )$$

wherein % ΔB_{S} represents the y-intercept on the calibration curve (the percent change in R_{ct} or Z_{mod} between the sensing electrode 2 without and with the clinical sample). Using the % ΔB_{B} of a blinded clinical sample (in which the concentration of specific markers is unknown) and experimentally derived B_{Φ} values (calculated using whole blood samples containing no specific markers), the true intercept value of the sensing biosensor (electrode 2 containing biotinylated aptamer) is calculated. Therefore, Equation 1 is modified as follows:

$$y = m x + ( {B}_{Φ} \times {B}_{B} ) \qquad ( Equation - 7 )$$

This will provide the values for the y-axis. Thus, knowing the ‘y’ axis values from the electrode 2 and the true intercept combined with the slope, ‘m’ determined from the calibration curve, the unknown concentration of the specific marker can be determined. In this fashion, the two-electrode approach is used to nullify the adverse effects of any non-specific adsorption of proteins, biological factors, and biological molecules giving the true value of the unknown markers present in the blood.

In certain embodiments, according to the invention, aptamers or antibodies are a form of biological detection (sensing) agent that are utilized to detect whether there exist certain analytes/biomarkers within a subject fluid sample. The term “aptamer” as used herein, refers to an oligonucleotide or oligonucleotide chain that has a specific and selective binding affinity for an intended target compound or molecule (e.g., analyte) of interest. The aptamer or antibody is capable of forming a complex with the intended target compound or molecule of interest. The complexation is target-specific in the sense that other materials which may accompany the target, do not complex to the aptamer or antibody. It is recognized that complexation and affinity are a matter of degree; however, in this context, “target-specific” means that the aptamer or antibody binds to the target with a much higher degree of affinity than it binds to contaminating materials. As used herein, the term “binding” refers to an interaction or complexation between the target compound or molecule of interest and the aptamer or antibody. Aptamers or antibodies are used in diagnosis by employing them in specific binding assays for the target compound or molecule of interest.

As used herein, “biomarkers” refer to naturally occurring or synthetic compounds, which are a marker of a disease state or of a normal or pathologic process that occurs in an organism. The term “analyte,” as used herein, refers to any substance, including chemical and biological agents that can be measured in an analytical procedure. The term “bodily fluid”, as used herein, refers to a mixture of molecules obtained from a patient. Bodily fluids include, but are not limited to, exhaled breath, whole blood, blood plasma, urine, semen, saliva, lymph fluid, meningeal fluid, amniotic fluid, glandular fluid, sputum, feces, sweat, mucous and cerebrospinal fluid. Bodily fluid also includes experimentally separated fractions of all the preceding solutions or mixtures containing homogenized solid material, such as tissues and biopsy samples. According to the invention, biomarkers and/or analytes are detectable in bodily fluid, such as, but not limited to, a minute volume of blood.

An “array” is an intentionally created collection of molecules. The molecules in the array can be identical or different from each other.

The biosensors of the invention include a substrate having a conductive material interface applied or deposited onto the substrate. The conductive material interface is composed of or structured from various such materials known in the art. In certain embodiments, the conductive material interface includes platinum wires. In certain embodiments, the platinum wires are in the form of a multi-array of vertically aligned platinum wires and, optionally, cast or embedded in an epoxy substrate. For instance, one end of the platinum wire is embedded in the epoxy substrate and an opposite other end is exposed as an outer surface. The surface of the conductive material interface is then functionalized.

There are various conventional mechanisms for functionalizing the platinum wires including, but not limited to, adsorbing a binding material thereon. Non-limiting examples of suitable binder materials include immobilization agents such as avidin, streptavidin, and neutravidin. In certain embodiments, neutravidin is preferred. FIGS. 1a-1d illustrate the adsorption of avidin onto the platinum conductive material interface for the baseline and fully functionalized functional sensing biosensors. Further, as shown in FIGS. 1a and 1c, biotin is attached to the avidin for the baseline biosensor, whereas biotinylated aptamer or antibody is attached to the avidin for the fully functionalized functional sensing biosensor (as shown in FIGS. 1b and 1d). Binding of biotin only for the baseline biosensor ensures that only interference from the biological sample is detected, and there is no detection of the target antigen by the baseline biosensor. Whereas binding of the biotinylated aptamer for the fully functionalized functional sensing biosensor ensures that the target antigen is detected, in addition to interference, from the biological sample. The process of biotinylation generally includes covalently attaching biotin to a protein, nuclei acid or other molecule. Biotin is known to bind to avidin with high affinity. The aptamer or antibody can be biotinylated chemically or enzymatically using conventional processes and apparatus. The binding material is selected based on its capability to bind the particular and specific aptamer or antibody.

Prior to application or deposition of the binding material onto the conductive interface material, optionally, the surface of the conductive interface material is treated with a thiol-based compound, such as, an aminothiol, including but not limited to, cysteamine and/or glutaraldehyde (as shown in FIGS. 1a-1d for the baseline biosensor and the sensing biosensor). The immobilization agent, such as, avidin, is then adsorbed thereon.

For the baseline biosensor, biotin is directly or indirectly attached or deposited onto the conductive material interface and for the fully functionalized functional sensing biosensor, a biological sensor agent such as an aptamer or antibody is directly or indirectly conjugated to the conductive material interface. The biotin and/or biological sensor agent is indirectly attached by employing linker molecules, such as, but not limited to, proteins.

The aptamer or antibody for binding to the avidin is selected based on its capability to interact with the specific target biomarker. Non-limiting examples of suitable aptamer include biotinylated aptamer selected specifically for cardiac biomarkers, such as, but not limited to, those described herein, for example, BNP and TnT. Thus, the avidin is immobilized on the surface of the platinum wires and the biotin and biotinylated aptamer or antibody attaches to the avidin for the baseline biosensor and sensing biosensor, respectively.

In certain embodiments, self-assembled monolayers (SAM) which are molecular assemblies of molecules or proteins that form ordered domains spontaneously on surfaces via adsorption, are utilized to tether the aptamer or antibody (biological detection element) to the platinum wires (material interface) to maintain a connection between these two elements that can be transduced into a readable output. SAMs can be either simple (consisting of few components) or complex (consisting of multiple components), with each of the components playing a role in functionalizing the surface or the biological detection element to promote binding.

In certain embodiments, the multi-array of platinum wires is embedded in an epoxy substrate, the surface of the epoxy substrate is polished and the wires on the surface of the epoxy substrate are treated with avidin followed by biotinylated aptamer or antibody. The biotinylated aptamer or antibody can include biotinylated proteins. In certain embodiments, the biotinylated aptamer or antibody is selected based on its ability to interact with cardiac biomarkers. Thus, BNP and TnT aptamer may be selected to interact with BNP and TnT biomarker, respectively. These cardiac biomarkers are released into bodily fluids, e.g., blood, for example, as a result of myocyte stretching or injury or death.

In certain embodiments of the invention, the fully functionalized functional sensing biosensors of the invention include at least one biological sensor agent and at least one signaling agent wherein the biological sensor agent(s) and signaling agent(s) together provide a means for detecting, signaling, and/or quantifying target compounds of interest in bodily fluids, such as, blood. The biological sensor agent is selected for its ability to specifically, and also selectively interact with and bind to (only) the target analyte/biomarker molecules. In accordance with the invention, the biological sensor agent is attached to the surface of a conductive material interface. The biological sensor agent is introduced by functionalization of the surface of a conductive material interface. The conductive material is a multi-array of vertically aligned platinum wires cast in an epoxy substrate. The biological sensor agent is an aptamer- or antibody-linked protein immobilized on a surface of the conductive material interface. The aptamer or antibody is conjugated to a signaling agent, e.g., the electrochemical impedance signal. The signaling agent is detectable under preselected conditions, e.g., after aptamer or antibody binding to the analyte/biomarker of interest. In accordance with the invention, signaling is related to a change in impedance, upon binding of the aptamer or antibody with the analyte/biomarker of interest. An end of the platinum wires provides a point of contact for an electrochemical impedance signal to be transduced to an interpretable read-out value.

In certain embodiments, the invention utilizes platinum wire as a conductive material interface and platform for a biosensing surface. The immobilized biological sensor is applied to the platform. The immobilized biological sensor includes the biotin for the baseline biosensor and the aptamer or antibody, e.g., biotinylated aptamer or biotinylated antibody, for the fully functionalized functional sensing biosensor, and the immobilization agent. Furthermore, the signaling agent includes the electrochemical impedance signal. The aptasensor is tailored to detect various markers predictive of disease, e.g., cardiovascular disease (CVD), in bodily fluids, primarily, but not limited to, blood, to determine the risk state of a patient. The sample of bodily fluid can be a minute volume, such as, for example, a few drops (e.g., about 1-5 drops) of blood, and the determination can be obtained in a relatively short period of time, such as, for example, about few or several minutes to five minutes. The cardiac markers can include C-reactive protein, Creatinine Kinase, TroponinT (TnT), Myoglobin, IL-6, IL-18, Brain natriuretic Peptide, and D-Dimer. Brain Natriuretic peptide (BNP) is an indicator of myocyte stress and Troponin-T (TnT) is an indicator of myocyte injury. The multi-array aptasensors in accordance with the invention provide portable, point-of-care (POC), on-demand devices that can be utilized in the absence of expensive equipment and highly trained professionals to assess levels of cardiac markers in the blood at the patient's bedside, for example, within a short period of time, such as, several minutes. Since the use of these aptasensors do not require much skill or training, they represent a simple and facile mode of detection.

Further, known impedimetric devices utilize antibodies and enzymes as detection elements, and platinum is used in tandem with other material interfaces such as carbon nanotubes/nanocomposites, graphene, chitosan, silica, polymers, or gold. In contrast, the multi-array aptasensors in accordance with this invention utilize platinum alone as the material interface.

Synthesis of multi-array aptasensors, in accordance with this invention, includes the use of appropriate linkers and proteins to immobilize marker-specific aptamers to the surface of the platinum wires. The platinum wire arrays, e.g., vertically aligned, are embedded in an epoxy mold, e.g., in a circular fashion or pattern, and the surface of the epoxy mold is polished, e.g., to approximately 50 nm, for surface exposure. The surface then can be treated with a thiol-based compound, such as, an aminothiol, including but not limited to, cysteamine and/or glutaraldehyde. An immobilization agent, such as, avidin, is adsorbed thereon. One or more aptamers is conjugated with biotin. The biotinylated aptamers for the target markers interact with the immobilization agent to develop the biosensing surface. Application of the treating agent and the immobilization agent, and interaction of the biotinylated aptamers can be carried out in a sequential manner, to develop the biosensing surface. As previously described, aptamers are essentially similar to antibodies, except in that, aptamers are oligonucleotide sequences that are highly specific for their designated antigen. However, unlike antibodies, aptamers can undergo denaturation and renaturation. The aptasensors can therefore be regenerated in the presence of certain solvents or other electrical or electrochemical signals, thus providing a reusable and regenerative sensor for potentially continuous use rather than one-time detection (as in commercially known glucose sensors). Thus, aptamers are more robust with a longer shelf-life and more importantly, allowing for aptasensors to be reusable rather than only a one-time, single-use assay.

In certain embodiments, an aptamer-based biosensor includes vertically aligned platinum electrode wires. Cysteamine (C), glutaraldehyde (G), and Streptavidin/NeutrAvidin (N) self-assembled monolayers (SAM) are first formed on the vertically aligned platinum (VAP) wires using the Layer by Layer (LbL) method. SAMs tether the biotin-based aptamer (biological detection element) to platinum, Pt maintaining contact between the two elements for transducing to a readable output. For use in testing the aptamer-based biosensor, a calibration curve is prepared for the human serum and whole blood samples.

An electrochemical sensor can be used to measure a change in output of a sensing element caused by chemical interaction of a target marker on a sensing element of the fully functionalized functional sensing biosensor. In accordance with the invention, electrochemical impedance spectroscopy (EIS) is the technique, e.g., sensor agent, utilized to characterize the surface of an aptasensor at various stages of development. EIS is a highly sensitive and label-free technique that allows for changes in electrochemical impedance resulting from the binding of the aptamer to the antigen. The electrochemical impedance can be transduced to a read-out value. Thus, the aptasensor is capable of electrochemically detecting biomarker concentrations that are present in a minimal amount of blood by measuring the impedance changes that occur upon the binding of antigens to the aptasensor. The impedimetric detection of the cardiac biomarker can be performed within minutes, and the aptasensor can be reused for this purpose multiple times.

In accordance with the invention, vertically aligned modified platinum wire-based aptasensors are provided for the impedimetric detection of cardiac markers. The aptasensors are synthesized by casting upright platinum wires in epoxy. The wires can be cast in various configurations and patterns. In certain embodiments, the wires are cast in a circular pattern. The diameter of the wires may vary and can range from about 0.25 mm to about 1.0 mm. In certain embodiments, the diameter is about 0.25 mm or about 0.5 mm or about 1.0 mm.

One end of the wire is cast in the epoxy and the opposite end has an immobilized aptamer attached thereto. Thus, the wires are utilized for functionalization and establishing electrical connection.

The resulting platinum electrodes are polished using polishing media, such as, but not limited to, silicon carbide (SiC), of varying different grit sizes and functionalized to bind the cardiac biomarker-specific aptamers to the surface. The surface roughness can vary and, for example, the polishing grit size, can range from about 320 grit (e.g., about 50 μm) to about 2400 grit (e.g., about 50 nm). In certain embodiments, the grit size is about 320 grit or about 1200 grit (e.g., about 5 μm) or about 2400 grit. It is contemplated and understood that the impedimetric devices can be tested against various clinically relevant concentrations of cardiac biomarker to determine the ideal wire diameter and polishing grit.

In certain embodiments of the invention, the aptasensors utilize 0.5 mm-diameter wires polished to about 1200 grit (e.g., 5 μm) size.

Electrochemical impedance spectroscopy (EIS) can be employed as a mode of impedimetric detection for the one or more cardiac biomarkers.

Impedimetric biosensors provide one or more of the following features and advantages as compared with known biosensors of CVD: highly sensitive, accurate, reproducible, low cost, allow for rapid analysis and miniaturization, and label-free, thus significantly reducing the complexity of biosensor development.

The biosensors developed in accordance with the invention may function as ex-situ biosensors. A portable (e.g., point-of-care (POC), on-demand) device, such as, a handheld device, may be developed. There are various mechanisms that are known in the art to produce a handheld device that may be employed with the biosensors and are suitable for use with the biosensors of this invention. In certain embodiments, the electrochemical impedance signal is transduced to a read-out value, and the read-out value is displayed on a handheld device. The handheld device can be an electronic device. Alternatively, the handheld device can include, for example, a test strip similar to conventional glucose sensors which are known in the art. There is typically a corresponding standard chart or key used to interpret the results displayed on the test strip. In these embodiments, the test strip is contacted with a patient bodily fluid sample, such as by applying the sample, e.g., a few drops, to the test strip or by dipping/immersing the test strip into the bodily fluid sample. The test strip is then visually observed or inspected to determine whether there is a visible change, such as a change in color, based on its contact with the sample. The mere presence of a visual change, such as color change, is indicative of a change of electrochemical impedance, e.g., binding of the aptamer or antibody in the test strip with the cardiac biomarkers in the bodily fluid sample, and therefore, the presence in the sample of the cardiac biomarkers of interest. Further, the corresponding key or chart can include varying degrees or intensity of change. The degree or intensity of visual change on the test strip is correlated to a particular quantitative amount of the electrochemical change and corresponding level of cardiac biomarkers of interest in the sample. Similarly, the absence of a visual change on the test strip is indicative of the absence of the cardiac biomarkers of interest in the patient bodily fluid sample.

For example, in accordance with certain embodiments of the invention, a bodily fluid sample, such as blood, is obtained or removed from a patient. Further, the sample can be obtained or removed by the patient. At least a portion of the sample is deposited on the test strip and within a given time-period, e.g., seconds or a few minutes, a change in color of at least a portion of the test strip is visually observed based on the cardiac biomarkers in the sample interacting with the test strip, e.g., biosensor. The particular specific color and/or the intensity of the color change is compared and matched with a key to determine the level of the cardiac biomarker, e.g., BNP and/or TnT, in the sample. Based on the visible change of the biosensor, the presence or absence or particular, specific concentration of the cardiac biomarker is determined efficiently and accurately. The response time may be in minutes or even seconds, and the results can be obtained by the patient in a domestic setting, without the need for medical personnel, laboratory equipment, emergency, and a medical facility.

Therefore, impedimetric biosensors in accordance with the invention are ideal portable, e.g., point-of-care (POC), on-demand, diagnostics that can be used, for example, at bedside, in ambulances, by paramedics or even during clinical visits as a useful screening device for the detection of cardiac biomarkers and therefore, the diagnosis of CVD conditions.

Further, in accordance with the invention, impedimetric biosensors exhibiting the following attributes are provided: (i) re-usable aptamer- or antibody-based electrochemical assay; (ii) multiple cardiac biomarker detection in a single setting; and (iii) amenable to hand-held model translation.

Point-of-care handheld aptasensors in accordance with the invention allow patients to frequently detect and measure their cardiac biomarkers and therefore, assess their individual CVD risk and to monitor how different lifestyle changes can reduce this risk. In addition, the aptasensors are inexpensive, e.g., comparable in price to blood-based glucose biosensors that are currently commercially available. Further, existing insurance codes for glucose biosensors and cardiac biomarker testing could be readily applied to aptasensors for full or partial reimbursement of the cost. Thus, patients can affordably, routinely, accurately, reproducibly, and rapidly detect and measure their cardiac biomarkers.

In an emergency room setting, significant minimization of turn-around time may be realized, resulting in more efficient allocation of resources, and providing more effective care for patients. For example, decreasing the time of diagnosis can reduce the time required to make an admission decision and therefore, ensure administration of rapid care to the patient. In addition, decreasing the time of diagnosis may also ensure that patients suffering from less severe conditions are not allocated more expensive, redundant resources.

In certain embodiments, the aptasensors can be tailored with a wireless chip to allow for wireless transmission of biomarker levels to a patient's electronic health records. Thus, reducing the amount of paperwork necessary and allowing the physician to directly view trends in the levels of biomarkers and detecting early a precarious patient CVD situation. In an overall health-care system setting, the aptamers may eventually allow for the replacement of antibodies with aptamers for immunoassays.

It should be understood and realized that the embodiments described herein, and the examples provided below are for illustrative purposes only and that various modifications or changes in light thereof will be suggested to persons skilled in the art and are to be included within the spirit and purview of this application.

### EXAMPLES

### Example 1

A coverage study was conducted wherein various concentrations of aptamer and biotin were bound to a Neutravidin-functionalized electrode to determine: (1) the saturation point of Neutravidin with biotin and biotinylated aptamer; (2) whether the amounts of Neutravidin and aptamer from the covered section were sufficient to ensure complete binding/coverage; and (3) whether reverse calculations ensured that the determined bound amounts of Neutravidin biotin, and biotinylated aptamer were reasonably correct. The results demonstrated that all the biotin sites on Neutravidin were saturated with biotin for the base biosensor (B_{B}) and aptamer for the fully functionalized functional sensing biosensor (B_{S}).

Upon depositing a biological sample (with no BNP or TnT antigen present) on both a baseline biosensor and a fully functionalized functional sensing biosensor, the baseline biosensor percent change from the initial value was higher than the sensing biosensor value, which was inferred as being due to the steric hindrance from the aptamer. As depicted in FIGS. 1a and 1c, Neutravidin (Avidin) bound by biotin only allowed for more exposure of the Neutravidin surface to the biological sample, whereas (as shown in FIGS. 1b and 1d) Neutravidin (Avidin) bound by biotinylated aptamer was less exposed due to the steric hindrance presented by the aptamer itself. The correlation between the two biosensors is represented by B_{Φ}:

B_{Φ}=(%ΔB_{S})/(%ΔB_{B}) (Equation-2)

wherein % Δ B_{S}=% Δ R_{ct} or Z_{mod} between the biosensing electrode (biotinylated aptamer, B_{S}) with and without the biological blood/serum/fluids and the biological blood/serum/fluids does not contain any BNP or TnT protein; and % Δ B_{B}=% Δ R_{ct} or Z_{mod} between the baseline electrode (biotin only, B_{B}) with and without the biological blood/serum/fluids and the biological blood/serum/fluids does not contain any BNP or TnT protein. Therefore, B_{Φ} is essentially the ratio between the y-axis intercept for the B_{S} biosensor to the B_{B} biosensor, where the y-intercept is presented as % ΔR_{ct} or Z_{mod} (the percent change between the signal of the biosensors with and without whole blood). Based on the electrical resistivity equation and using experimentally measured B_{B} and B_{S} values from rat blood, it was demonstrated that B_{Φ} represents the coverage of Neutravidin by the biological sample. The calculation also led to the conclusion that B_{Φ} is represented as:

$${B}_{Φ} = {Area}_{exposed} ( {A}_{e} ) \div Total Area ( A ) = {A}_{e} \div A \qquad ( Equation - 3 )$$

Substituting Equation-2 with the layer thickness of the blood- and without blood-containing layers and solution resistivity resulted in Equation-3. B_{Φ} is also represented as follows:

$${B}_{Φ} = ( {B}_{B} - {B}_{N} ) / {B}_{S} \qquad ( Equation - 4 )$$

wherein B_{N} is the signal measured from the baseline of the biotin only electrode without any biological fluids or bloods, B_{B} is the signal of the biotin only electrode in the presence of blood/serum/biological fluids, and B_{S} is the signal of the biotinylated aptamer electrode in the presence of blood/serum/biological fluids. The B_{S} signal was further normalized following the equation:

$${B}_{S}^{N} = ( {B}_{N} * {B}_{S} ) / {B}_{SB} \qquad ( Equation - 5 )$$

wherein B_{S}^{N} is the normalized signal of the biotinylated aptamer electrode in presence of blood/serum/biological fluids, and B_{SB} is the signal measured from the baseline of biotinylated aptamer electrode without any biological fluids or bloods.

To calculate the true intercept of the B_{S} biosensor using the B_{B} biosensor and the average B_{Φ} values, Equation-2 can be modified as follows:

$$\% Δ {B}_{S} = {B}_{Φ} \times \% Δ {B}_{B} \qquad ( Equation - 6 )$$

wherein % ΔB_{S} represents the y-intercept on the calibration curve (the percent change in R_{ct} or Z_{mod} between the biosensing electrode without and with the clinical sample). Using the % ΔB_{B} of a blinded clinical sample (in which the concentration of BNP and TnT present is unknown) and experimentally derived B_{Φ} values (which were calculated using whole blood samples containing no BNP or TnT), the true intercept value of the biosensing biosensor (a biosensor containing biotinylated aptamer) is calculated.

Therefore, Equation 1 is modified as follows:

$$y = m x + ( {B}_{Φ} \times {B}_{B} ) \qquad ( Equation - 7 )$$

The validity of the two-electrode correction approach as described above was tested against human blood and serum samples. Human serum/whole blood samples were acquired from the Cardiovascular Institute, University of Pittsburgh Medical Center (UPMC). The amount of BNP present in twenty unknown serum/whole blood samples were measured using ELISA assay (clinical gold standard for the determination of BNP concentration). These human serum and whole blood samples were used to obtain the sensor response utilizing the inventive biosensor system and different concentrations of BNP was determined using the calibration curves (shown in FIG. 2) in a range of 100 to 1200 pg/mL. Percent change represents the percent change of the signal (Rct) between the biosensor baseline (0.00 ng/mL) value and the respective concentration. The two-electrode correction method described above was used to negate any unspecific protein adsorptions on the sensor electrodes.

FIGS. 3 and 4 show the results obtained for BNP concentration measured using the two-electrode biosensing approach for the 20 unknown serum and whole blood samples. The results clearly demonstrate that the BNP values obtained using the developed inventive biosensor are very similar to the gold standard ELISA values. The value of BNP concentration in the whole blood (WB) is almost half of the serum as the volume of WB is twice than that of the serum. The volume of serum obtained after blood clotting is reduced by ˜50% in volume due to the removal of various cells and platelets in the form of solid clotting. The BNP values of WB samples obtained using the biosensor system and technology according to this invention follows the same trend obtained for the serum samples and therefore, confirms the use of this developed biosensor for both human WB and serum samples.

The biosensor platform is innovative and highly versatile, and the correction method described above, are clearly universal and can be applied for detection of various other biomarkers in the blood. Accordingly, the applicability of this correction method was further evaluated and tested for the detection of immunosuppression drugs (ISDs) such as Tacrolimus (TAC). Biotin-conjugated antibodies of Tacrolimus (TAC) were immobilized on a novel functionalized platinum surface that can detect minute changes in the electrochemical impedance values of the interaction of antibodies with the analytes. The results (see FIG. 5a) show that the optimal Pt wire diameter and surface polish of 0.5 mm and 5 μm provide linear calibration with excellent concentration correlation for TAC levels in mouse blood (known TAC concentration added to whole mouse blood) while demonstrating good precision and error margins. The results obtained show that the inventive biosensor is sensitive to clinically relevant TAC ranges, detecting TAC levels as low as 1 ng/ml and hence, clearly shows the applicability of the inventive biosensor for therapeutic drug monitoring (TDM) of ISDs. To further validate the TAC detection ability of the biosensor, three blinded samples of known TAC amounts dissolved in mouse blood but unknown to the researcher were analyzed. The obtained biosensor responses were corrected using the two-electrode approach discussed above to eliminate any non-specific adsorption, biofouling, and cross-sensitivity. FIG. 5b clearly shows the detection accuracy of the micro-sensing based active point of care (POC) immunosuppression tracking (MAP-IT) for these three blinded samples.

### Example 2

### 2.1 Single-Frequency Calculation

The optimal single frequency was calculated by comparing the bode plots for the optimal functionalization combination (PCGNB/T) antigen detection and determining the frequency at which the percent change in absolute impedance, known as Zmodulus (% Δ Z_{mod}) between concentrations correlated best with the concentrations themselves.

### 2.2 Aptamer Regeneration Strategy

Aptamer regeneration was explored utilizing two strategies-applied current (chronopotentiometry, which measures the voltage difference with an applied constant current over time) and applied voltage (chronoamperometry, which measures the current difference with an applied constant voltage over time). All chronopotentiometry and chronoamperometry experiments were performed using a Gamry series G Potentiostat (GAMRY PCI4-G300) in an electrolyte solution of 10 mM Trizma, 50 mM KCl, and 1.5 mM MgCl₂ in deionized water (which represents the buffer used for Polymerase Chain Reactions (PCR), thus implying that this buffer will not damage or interfere with the DNA aptamers).

### 2.3 Antigen Calibration Curves (Various Biological Samples)

In order to determine the antigen detection capabilities of the biosensor and the resistance of the biosensor to interference, various biological samples were tested. Concentrations of 0.2 ng/mL, 0.6 ng/ml, 1.0 ng/ml, and 2.0 ng/ml BNP, and 0.005 ng/mL, 0.01 ng/mL, 0.02 ng/mL, and 0.04 ng/ml of TnT were prepared in the following solutions—(1) PBS (the phosphate buffered saline (PBS) buffer that has been utilized for all testing so far), (2) Dulbecco's Eagle Medium (DMEM) containing 10% Fetal Bovine Serum, (3) Filtered Human Serum, and (4) five separate rat whole blood samples (R3, R36, R37, R38, R39) obtained Sprague Dawley Rats sacrificed after 4-16 weeks post-magnesium alloy (ZJ41) implantation. Concentrations were prepared in 25 μl volumes and stored at 4° C. until testing. Each concentration was tested on a different batch of electrodes (instead of successive concentrations on one set of electrodes) to avoid increased interference from biological substances present in the samples. Two concentration incubation times (30 seconds and 5 minutes) and two testing methods (single-frequency vs. frequency-range EIS) were employed for determining the antigen calibration curves.

### 2.4 Neutravidin Coverage

Two sets of electrodes were prepared as optimized above up to the Neutravidin stage. Upon Neutravidin binding, one set of electrodes was treated with varying concentrations of biotin (1 ng/μL, 5 ng/μL, 10 ng/μL, and 15 ng/μL prepared in PBS), while the other was treated with varying concentrations of BNP and TnT aptamer (0.1 μg/uL, 0.5 μg/uL, 1 μg/uL, and 1.5 μg/uL). Electrodes were tested after incubation with each concentration Each set of electrodes were graphically compared in Microsoft Excel to determine the approximate saturation point, and using the molar mass of each component, the mass bound was derived from the concentration saturation point.

### 2.5 Two-Electrode Approach

To perform the two-electrode approach for each sample above and subsequent samples, three sets of electrodes were prepared-one set with Neutravidin bound with biotin only (baseline biosensor, B_{B}), and two sets with Neutravidin bound with biotinylated BNP and TnT aptamer respectively (fully functionalized functional sensing biosensor, B_{S}). The rat whole blood samples from Section 2.3 were tested using the inventive biosensor method to obtain the BΦ correction values, and these correction values were then applied for future human serum testing described below.

### 2.6 Human Serum Testing

Ten clinically derived serum samples from patients implanted with Ventricular Assist Devices (VADs) were obtained from the Cardiovascular Institute, University of Pittsburgh Medical Center (UPMC). Of those ten samples, only eight were measured for BNP concentration values at UPMC via ELISA assay; to determine the efficacy of the biosensor, the eight samples with known concentrations of BNP were measured using the inventive two-electrode approach biosensing (Section 2.5) via the same four tests described in Section 2.3.

### 2.7 Statistical Analysis

Statistical analysis for antigen detection was performed using Microsoft Excel (Microsoft Office). Increases in Rct and Zmod between concentrations was compared between wire diameters and polishing grit (n=3 per concentration) using standard error (SE). Statistical analysis for comparing for BΦ and MΦ values was performed using Graphpad Prism 7 (Graphpad Software, Inc.). BΦ experimental and calculated values were compared using a 2-way ANOVA (factors were test type—Zmod or Rct for 30 s or 5 m—and evaluation type-experimental vs. calculated) with Sidak's post-hoc testing. MΦ experimental values were compared using a 1-way ANOVA with Tukey's post-hoc testing. Statistical analysis for comparing the obtained concentrations from different methods and corrections to the lab-value concentration were compared using a 2-way ANOVA (factors were test type and sample number) with Dunnet's multiple comparison's post-hoc testing. All graphical representations reflect mean+SE.

### 2.8 Results and Discussion

### Single-Frequency Calculation

One of the aspects of using EIS as the transducer is the rapidity of the assay (which takes approximately 2 minutes at the frequency range utilized). However, in order to make the assay even faster, the resultant Bode plots from the BNP and TnT antigen assays were compared and the single frequency was calculated at which both antigen detection assays demonstrated the highest correlation across the entire concentration range. A Bode plot is another interpretation of an EIS spectra, like a Nyquist plot, but instead of depicting impedance as real and imaginary Cartesian coordinates based off a parametric frequency response, a Bode plot separates the real and imaginary components as a function of frequency. Therefore, in a Bode plot, the impedance is graphed as the absolute impedance (Zmodulus, Zmod), which is essentially a scalar quality and represents magnitude, and the phase angle (Zphz), which represents the phase in a vector quality, as functions of frequency. When comparing the Bode and the Nyquist plots, it was observed that the y-axis of the Bode plots (the Zmod) matched the x-axis of the Nyquist plots (Z′), thus further validating that the Bode plots essentially reflect the real component of impedance. Based off the Bode plots, the highest change in Zmod across all the concentrations of BNP and TnT occurred between 100 Hz and 10 Hz. Beyond 10 Hz, there was observed, a slight drop in the change in Zmod, although that change had to be examined numerically rather than graphically. Therefore, the calibration curves across those frequencies were calculated and the frequency at which the calibration curve correlation coefficient was the highest was determined. At the frequencies between 100 Hz and 10 Hz, the changes in Zmod across all the concentrations were nearly similar (similar slopes); thus, the correlation coefficient was examined to assess differences between frequencies. Based off the calculations, it was determined that the optimal single frequency for rapid (15-30 s) testing for antigen detection was f=20 Hz, which demonstrated the highest correlation coefficient.

### Biosensor Regeneration

As previously disclosed, the use of aptamers can allow for the biosensor to be reused (regenerated) in such a way that only the aptamer unfolds, releases the antigen, and then refolds into its original configuration, thus allowing it to be reused for subsequent measurements. However, many of the regeneration strategies for aptamers require the use of weak acids, weak bases, detergents, or chaotropic reagents that can severely impact the impedimetric response of the biosensor. Therefore, a strategy for reducing the impact of regeneration on the impedimetric response was to use electrochemistry itself to manipulate the aptamer into unfolding and refolding into the current configuration. If feasible, the regeneration strategy could be utilized to account for background interference by depositing the sample on the surface and then only regenerating the aptamer itself, thus leaving the bound background interference intact.

BNP biosensors were prepared and then tested to obtain the baseline value (no antigen incubation or regeneration). The BNP biosensors were exposed to various voltages and currents for a variety of times to assess if the voltages or currents disrupted the intact biosensor system. Ideally, to use an applied voltage or current for regeneration, the current or voltage must not impact the system to ensure that any reduction or increase in signal is the result of antigen binding and antigen removal alone. Therefore, the % Δ Rct between the intact biosensor and the biosensor after an applied voltage or current was measured for a wide variety of times. A negative % Δ Rct would be indicative of a stripping event, in which the SAM layers were being stripped from the biosensor, while a positive % Δ Rct would be indicative of a charging event, in which the SAM layers were somehow storing charge and thus impacting the electrochemistry and kinetics of the system.

Both regeneration strategies were tested in PCR buffer, as this is the buffer traditionally used for polymerase chain reactions, during which DNA strands unzip, unfold, and then is replicated repeatedly. Therefore, it was determined that using the PCR buffer for the electrochemical testing would ensure that the DNA aptamers were not damaged in the process. However, with applied voltage, especially at higher magnitudes of voltage, it was observed that the biosensor layers themselves were being stripped off, even with a voltage application that lasted only 1 ms. With applied current, any current above −1.0 μA actually caused an increase in Rct, whereas lower magnitudes were, like similar to the applied voltage, too strong and removed the biosensor layers themselves. The increase in Rct may have been caused by accidentally charging the residues present in Neutravidin, or accidentally charging the aptamer itself as DNA can carry a current. However, as it could not go below 60 μs due to the constraints of the potentiostat, it was not evident whether an applied current or voltage for a smaller time frame could potentially provide the solution to regenerate the electrode. Application of a weak pulse of current for time duration lower than 60 μs could serve to prevent stripping all of the biosensor layers and only remove the outer analyte layer thereby enabling reuse of the biosensor. In addition, the measurements taken for this experiment were conducted immediately after application of the voltage or current. The aptamer may have needed time to reconfigure before the Rct values return precisely back to the original baseline.

### Biological Sample Antigen Testing

Using BNP and TnT biosensors, there were tested various different antigen concentrations (0.25 ng/ml-2.0 ng/mL BNP, 0.005 ng/ml-0.04 ng/ml TnT) in various different biological samples (PBS, DMEM+10% FBS, Human Serum (HS), and rat whole blood samples R3, R36, R37, R38, and R39). The antigen concentrations were tested using two methods-Single frequency impedance (Zmod at 20 Hz), and electrochemical impedance spectroscopy (Rct across 10,000 Hz-1 Hz) for two timepoints-30 s and 5 m. Then there was a comparison of the findings across samples for each method.

From these experiments, it was discovered that the bar graphs for PBS prepared antigens remained relatively constant across the different time points and measurement methods, but that the bar graphs for the other biological samples varied considerably. This variation was likely the result of bio-fouling/interference from the other proteins and factors present in the biological samples, especially in human serum and the rat whole blood samples. It was also observed that with the higher timepoints, in both methods, that the differences between the whole blood samples, especially R36, R37, and R38 vs. R3 and R39, were much larger, thus indicating that higher timepoints (5 m) not only demonstrated better sensitivity, but also amplified outliers like R3 and R39 from the general trend represented by R36, R37, and R38. In addition, R36, R37, and R38 actually fell within the same range as human serum for both BNP and TnT across all the methods and timepoints. Based on these experiments, it was determined that the most optimal method for antigen detection was Zmod, 30 s, as it reduced the amplification of outlier samples and was relatively consistent compared to the other samples across the board. In addition, the short time frame reduced the interference from all the factors present in serum and whole blood. However, the disadvantage of the Zmod, 30 s method is that 30 s is a very short time for antigen binding, which decreases the sensitivity considerably compared to the 5 m methods.

### Model #2 (BΦ Intercept Correction)

In this model, in order to correct each calibration curve's intercept, a two-electrode approach according to the invention (see FIGS. 1a-1d) was employed, where one electrode served as a baseline biosensor (B_{B}) and the other electrode served as a fully functionalized functional sensing biosensor (B_{S}). B_{B} was a baseline biosensor that was functionalized with biotin only instead of biotinylated aptamer after Neutravidin binding, thus ensuring that the biosensor cannot detect the target antigen and can only detect interference from the biological sample. On the other hand, B_{S} was functionalized with biotinylated aptamer, thus enabling the fully functionalized functional sensing biosensor to detect both the interference from the biological sample, but also the target antigen. Therefore, it is essential for all the biotin sites on Neutravidin to be bound by either biotin (in the case of B_{B}) or biotinylated aptamer (B_{S}). To ensure complete biotin site occupation, a coverage study was performed, where various concentrations of aptamer and biotin were bound to a Neutravidin functionalized electrode to determine (1) the saturation point of Neutravidin with biotin and biotinylated aptamer, (2) whether the amounts of Neutravidin and Aptamer were sufficient to ensure complete binding/coverage, and (3) whether reverse calculations ensure that the determined bound amounts of Neutravidin, biotin, and biotinylated aptamer were correct or appropriate.

### Coverage Study

First, there was determined the binding activity of biotin to Neutravidin, the molar masses of the aptamers, biotin, and Neutravidin, and the ratio of biotin to aptamer in the biotinylated aptamers.
Maximum binding activity of biotin to Neutravidin=14 ng biotin/1 μg
Neutravidin Concentration of Neutravidin in 1 μl→1 mg/ml=1 μg/μl
Biotin molar mass=244 g/mol
Neutravidin molar mass=60,000 g/mol
BNP biotinylated aptamer molar mass=22778.9 g/mol (22534.9 g aptamer+244 g biotin) Ratio of biotin: BNP aptamer molar mass=244 g: 22534.8 g=0.01
TnT biotinylated aptamer molar mass=22458.8 g/mol (22214.8 g aptamer+244 g biotin) Ratio of biotin: TnT aptamer molar mass=244 g: 22214.8 g=0.01
Four concentrations of biotin (1 ng/μl, 5 ng/μl, 10 ng/μl, and 15 ng/μl) and corresponding BNP and TnT aptamer concentrations (0.1 μg/μl, 0.5 μg/μl, 1.0 μg/μl, 1.5 μg/μl) were prepared, wherein—
   1.5 μg/μl aptamer—0.01×1.5 μg/μl—15 ng/μl biotin
   1.0 μg/μl aptamer—0.01×1.0 μg/μl—10 ng/μl biotin
   0.5 μg/μl aptamer—0.01×0.5 μg/μl—5 ng/μl biotin
   0.1 μg/μl aptamer—0.01×0.1 μg/μl—1 ng/μl biotin.

There was tested 1 μl of each concentration of aptamer (BNP and TnT) and biotin over three electrodes each, and then they were plotted against each other to determine the approximate saturation point for the aptamers and biotin, which was approximately 1.0 μg/mL aptamer and 10 ng/mL biotin, respectively, which indicates that 10 ng biotin and 1.0 μg aptamer was bound to the Neutravidin electrodes. Therefore, the amount of Neutravidin bound to the electrode is determined—

$$\frac{1 μg Neutravidin}{14 ng biotin} \times 10 ng biotin = 0.71 μg Neutravidin$$

Therefore, approximately 0.71 μg of Neutravidin was bound to the electrode surface. To determine whether these calculations were within reason, there was performed a reverse calculation using the molar masses to determine the number of moles bound.

$$0.71 μg Neutravidin = 7.1 \times 1 {0}^{- 7} g \times \frac{1 mol}{60 , 000 g} = 1.18 \times 1 {0}^{- 11} mol Neutravidin$$

$$10 ng biotin = 1. \times 1 {0}^{- 8} g \times \frac{1 mol}{244} = 4.10 \times 1 {0}^{- 11} mol biotin$$

$$1 μg BNP aptamer = 1. \times 1 {0}^{- 6} g \times \frac{1 mol}{22778.9 g} = 4.39 \times 1 {0}^{- 11} mol BNP aptamer$$

$$1 μg TnT aptamer = 1. \times 1 {0}^{- 6} g \times \frac{1 mol}{22458.8 g} = 4.45 \times 1 {0}^{- 11} mol TnT aptamer$$

It was then determined whether the molar ratios of aptamer and biotin to Neutravidin were approximately 4 (representing the tetrameric nature of Neutravidin, which can bind 4 biotin per protein)
Biotin: Neutravidin=4.10×10⁻¹¹ mol: 1.18×10⁻¹¹ mol=3.5
BNP Aptamer: Neutravidin=4.39×10⁻¹¹ mol: 1.18×10⁻¹¹ mol=3.7
TnT Aptamer: Neutravidin=4.45×10⁻¹¹ mol: 1.18×10⁻¹¹ mol=3.8.

Therefore, as all three molar ratios were reasonably close to 4, the calculations could be deemed correct, and it was assumed that all the biotin sites on Neutravidin were saturated with biotin (BB) and aptamer (BS).

### BΦ and MΦ Corrections

The equation was further modified by creating a slope correction factor known as MΦ, where,

$$M Φ = Calculated Value / True Value \qquad ( Equation 6 - 7 )$$

Therefore, incorporating the slope correction factor into Equation 5 as derived from FIG. 6a, as follows:

$$y = ( m \times M Φ ) x + ( B Φ \times BB ) \qquad ( Equation 6 - 8 )$$

This equation can now account for the drop interference by increasing the sensitivity of the calibration curve equation (which, as there is less interference in serum, the biosensor would be more sensitive). Then Equation 6-8 was used to recalculate the measurements (FIG. 6b).

Using Model #3, with both MΦ and BΦ corrections, it was observed that for most of the methodologies, but especially for 30 s-Zmod, that the calculated values are not significantly different from most of the true values. The exceptions are VAD119 and VAD122, but note that both samples have a true value higher than 2.0 ng/mL, which is already extremely high (and for both samples, the biosensors register between the 1.5-2.0 ng/mL range, which is also considered high values). Therefore, if the biosensor was solely used as a rule-in or rule-out CHF, cardiovascular heart failure method, the biosensor would already meet the requirements, especially as it is fairly accurate in the lower concentration ranges.

For Model #3-Bland Altman analysis showed that, while the bias values were not particularly low (0.2-0.4), the bias values across all four methods were comparable. The confidence intervals were still rather large, though there was observed excellent clustering of values near the bias lines, especially for the Zmod tests. However, VAD 119 (which had a very high concentration of BNP antigen, above 3 ng/mL) remained a consistent outlier. Removal of VAD 119 from consideration immediately dropped the bias values to 0.1 for all four methods, the confidence intervals became much smaller as well, although still not as small as desired. This was most likely due to the fact, that although VAD 119 was the main outlier, the biosensor also struggled to accurately detect the concentrations above 2.0 ng/mL for VAD 122 and VAD 125. Removal of these two points dropped the 30 s-Zmod bias value to nearly 0, and dropped the confidence interval to a mere ±0.1 ng/mL, which is excellent for differentiating between levels of BNP (not necessarily good for determining the actual concentration of BNP antigen present).

Therefore, using Model #3 with MΦ and BΦ corrections, especially for the 30 s-Zmod method, there was achieved a biosensor that could detect concentrations below a 2.0 ng/mL range relatively well, and could differentiate between BNP levels or ranges very well.

### CONCLUSIONS

Both BNP and TnT biosensors in biological samples (specifically, rat whole blood) were tested to determine an average calibration curve for future unknown sample detection (Model #1—No correction). However, the intercepts in the individual calibration curves were highly variable, so there was developed a two-electrode model to correct the calibration curve intercept for each individual sample (Model #2—BΦ Corrections). When both Model #1 and Model #2 were utilized against human serum samples with a known value of BNP only, it was discovered that neither Model was sufficient to accurately detect the concentrations of BNP due to the fact that, there was used a model developed for whole blood against human serum. Whole blood is typically drawn from the patient without any processing (although for longer storage, anticoagulants are utilized to prevent the blood from clotting). Whole blood contains red blood cells, white blood cells, platelets, proteins, antibodies, enzymes, salts, and plasma. When whole blood is centrifuged at a high speed (2500 RPM for 15 minutes), the plasma separates from all the cells and platelets, but still contains clotting factors, proteins, antibodies, enzymes, and salts. If whole blood is allowed to clot and is then centrifuged, the blood serum separates, which not only removes all the cells and platelets, but also removes any factors or proteins involved in coagulation, thus leaving behind a solution containing only proteins, antibodies, enzymes, salts, and water. These differences between whole blood and serum demonstrate that in a whole blood sample, there are more interfering factors that could bind to the biosensor than in serum, thus reducing the sensitivity of the biosensor and leading to calibration curves with lower slopes. Therefore, Model #3 (BΦ Corrections and MΦ Corrections) was created, which corrected the slope for sensitivity to human serum, and Model #3 could accurately detect all BNP concentrations under 2.0 ng/mL, which is an acceptable clinical range, especially for a rule-in/rule-out CHF method. Therefore, based off this pilot study, the novel biosensor shows immense potential for success in future studies.

### Example 3

### Testing of Biosensors Against Clinically Obtained Whole Blood Samples to Evaluate the Effectiveness as a Potential Ex-Situ CVD Screening Device

Prior to testing the human whole blood and serum samples containing clinically relevant BNP levels, the calibration curve for the human serum and whole blood samples is prepared. In order to determine the complete antigen detection capabilities of the biosensor and the resistance of the biosensor to any interference from non-specific adsorption of myriad proteins, various biological samples of serum and whole blood samples from unknown patients were tested. Concentrations of 0.1 ng/ml, 0.3 ng/ml, 0.6 ng/mL, and 0.9 ng/mL and 1.2 ng/mL BNP were respectively prepared in the following (1), human serum and (2) human whole blood sample solutions. The calibration curves were prepared following two different methods described below.

Method 1: This method involves the testing of all the concentrations on the same batch of electrodes after successive addition of each concentration and washing steps. The detailed procedures followed are given below.
i) Electrode functionalization: Vertically aligned Pt electrodes were treated with each functionalized layer. Electrodes were first treated with 1 μL of 20 mg/mL cysteamine prepared in de-ionized water for 30 min at room temperature, followed by 1 μL of 25% glutaraldehyde in de-ionized water for 15 min at room temperature for thiolation and carboxylation of the Pt-surface. The surface was then treated with 1.5 μL of 1 mg/mL Neutravidin prepared in 10 mM phosphate buffer solution (PBS) for 30 min at room temperature, followed by incubation with 1480 μg/mL biotinylated BNP aptamer for 30 min at room temperature. During functionalization, the electrodes were washed with de-ionized water for 30 sec and air dried before adding each subsequent layer. The functionalized electrodes were then stored in electrolyte solution at room temperature overnight.
ii) Antigen testing: Five concentrations of BNP were prepared by adding known amounts of BNP to human serum and human whole blood samples that originally contain very little or no BNP. Each concentration selected falls within the clinical range representing low to high risk for cardiovascular disease (CVD). The BNP-aptamer biosensors were thus successively treated with 1 μL of 0.1 ng/ml, 0.3 ng/ml, 0.6 ng/ml, and 0.9 and 1.2 ng/ml concentrations, respectively to develop a calibration curve. Each concentration was added after 30 sec of washing and air-drying steps. EIS measurements were then taken after each antigen incubation to obtain the calibration curves. All the antigen binding steps were conducted at room temperature to examine the rapid detection capability of the biosensor. The biosensor testing was performed for the above antigen concentrations using electrochemical impedance spectroscopy (Rct across 10,000 Hz-1 Hz) following 5 min incubation.

FIG. 7a shows the calibration curve obtained for detecting BNP in human serum and FIG. 7b shows the corresponding calibration curve obtained for detecting BNP in human whole blood.

Method 2: In Method 2, each concentration was tested on a different batch of freshly prepared electrodes (instead of using the same set of electrodes for testing successive concentrations of the antigen) for achieving better accuracy and also avoiding increased interference from any biological substances present in the human serum and whole blood samples. The detailed procedures involved in this method is provided below.
i) Electrode functionalization: The vertically aligned Pt electrodes were treated with each functionalized layer. Electrodes were first treated with 1 μL of 20 mg/mL cysteamine prepared in de-ionized water for 30 min at room temperature, followed by 1 μL of 25% glutaraldehyde in de-ionized water for 15 min at room temperature for thiolation and carboxylation of the Pt-surface. The surface was then treated with 1.5 μL of 1 mg/mL Neutravidin prepared in 10 mM phosphate buffer solution (PBS) for 30 min at room temperature, followed by incubation with 1480 μg/mL biotinylated BNP aptamer for 30 min at room temperature. During functionalization, the electrodes were washed with de-ionized water for 30 sec and then air dried before adding each subsequent layer. The functionalized electrodes were stored in electrolyte solution at room temperature for overnight.
ii) Antigen testing: Each concentration of BNP was prepared by adding known amounts of BNP to human serum and human whole blood samples that originally contained little or no BNP. Each concentration was then added to a different batch of fully functionalized electrodes and EIS measurements were then taken after antigen incubation to obtain the calibration curves. All the antigen binding steps were conducted at room temperature for 5 minutes to examine the rapid detection capability of the fully functionalized cardiosense biosensor. The biosensor testing was performed for the antigen concentrations using electrochemical impedance spectroscopy (Rct across 10,000 Hz-1 Hz). FIGS. 8a and 8b show the calibration curves obtained using Method 2 for detecting BNP in human serum and human whole blood samples, respectively.

### Example 4

The inventive biosensor system was used to determine the concentration of brain natriuretic peptide (BNP) in blinded unknown human blood and human serum samples. A total of 15 blinded human whole blood and human serum samples were tested using the inventive biosensor platform utilizing the novel two-electrode detection assembly overcoming any biofouling interference as well as any non-specific adsorption from other electrolytes, proteins and signaling molecules that are present in normal blood samples. Excellent correlation and agreement were obtained with the standard enzyme linked immunosorbent assay (ELISA) measurements. FIG. 9a shows data for the serum samples and FIG. 9b shows data for the whole blood samples.

We claim:

1. A biosensor system to detect biomarkers of interest in a patient and reduce or preclude non-biomarker interference, comprising:
a baseline biosensor, comprising:
an electrically conductive material interface having a surface,
an immobilization agent applied or deposited onto the surface of the conductive material interface; and
biotin conjugated with the immobilization agent;
a sensing biosensor, comprising:
a conductive material interface having a surface, and
a biological sensor agent applied to the surface of the conductive material interface, the biological sensor agent comprising:
an immobilization agent; and
at least one aptamer or antibody selected to interact with the immobilization agent and selected to bind with the biomarkers of interest;
a signaling agent comprising an electrochemical impedance signal generated by binding of the aptamer or antibody with the biomarkers of interest; and
a bodily fluid sample derived from the patient and in contact with the biotin of the baseline biosensor and at least one aptamer or antibody of the sensing biosensor,
wherein the baseline biosensor detects interference in the bodily fluid sample, and the sensing biosensor detects a presence of the cardiac biomarker of interest in the bodily fluid sample; and
a correction applied to the baseline biosensor and the sensing biosensor to reduce or preclude non-biomarker interference from the bodily fluid sample.

2. The system of claim 1, wherein the bodily fluid sample is selected from blood, serum, plasma, and urine samples of animals or humans.

3. The system of claim 1, wherein the biomarkers are selected from C-reactive protein, Creatinine Kinase, Creatinine Phosphokinase, TroponinT, Myoglobin, IL-6, IL-18, Brain Natriuretic Peptide, and D-Dimer, immunosuppression drugs, Tacrolimus; Sirolimus, Tau protein, glial fibrillary acidic protein and breakdown products (GFAP-BDP) and ubiquitin C-terminal hydrolase L1 (UCH-L1).

4. The system of claim 1, wherein the immobilization agent is a binding agent.

5. The system of claim 4, wherein the binding agent is selected from the group consisting of avidin, streptavidin, neutravidin and mixtures thereof.

6. The system of claim 1, wherein the conductive material interface comprises a multi-array of vertically aligned platinum wires.

7. The system of claim 1, wherein the aptamer or antibody is effective to impedimetrically detect simultaneously a plurality of biomarkers in the bodily fluid sample.

8. The system of claim 6, wherein the multi-arrays of vertically aligned platinum wires are arranged in a circular configuration.

9. A biosensing method of detecting biomarkers of interest in a bodily fluid sample of a patient and to reduce or preclude non-biomarker interference, comprising:
obtaining the bodily fluid sample from the patient;
forming a detection device, comprising:
a baseline biosensor, comprising:
a conductive material interface having a surface,
an immobilization agent applied or deposited onto the surface of the conductive material interface; and
biotin conjugated with the immobilization agent;
a sensing biosensor, comprising:
a conductive material interface having a surface, and
a biological sensor agent applied to the surface of the conductive material interface, the biological sensor agent comprising:
an immobilization agent; and
at least one aptamer or antibody selected to interact with the immobilization agent and selected to bind with the biomarkers of interest;
contacting the biotin of the baseline biosensor and the aptamer or antibody of the sensing biosensor with the bodily fluid sample;
generating an electrochemical impedance signal for each of the baseline biosensor and the sensing biosensor; and
applying a correction to the sensing biosensor based on the baseline biosensor to reduce or preclude non-biomarker interference from the bodily sample.

10. The method of claim 9, wherein the electrochemical impedance signal is transduced to a read-out value.

11. The method of claim 10, wherein the electrochemical impedance signal is connected to a portable device such as a hand-held device that is effective to display the read-out value.

12. The method of claim 9, wherein the detection device is in the form of a test strip and the method, comprises:
contacting the bodily fluid sample with the test strip;
assessing a visual change to the test strip;
correlating the visual change with a chart or key; and
based on said correlating, determining if the visual change is indicative of the presence of a change in electrochemical impedance and the presence of the cardiac biomarkers in the bodily fluid sample.

13. The method of claim 12, wherein the visual change is a color change.

14. The method of claim 9, wherein the electrochemical impedance signal is generated as a result of the biomarker of interest interacting with the aptamer or antibody.

15. The method of claim 9, wherein prior to applying the immobilization agent, the surface of the conductive interface material is treated with a thiol-based compound.

16. The method of claim 15, wherein the thiol-based compound is an aminothiol selected from cysteamine and/or glutaraldehyde.
